(19) United States
(12) Patent Application Publication
(10) Pub. No.: US 2010/0055703 A1
(43) Pub. Date: Mar. 4, 2010

(54) Organism-Specific Hybridizable Nucleic Acid Molecule

(75) Inventors: Marco Thines, Ostfildern-Ruit (DE); Frank Braendle, Stuttgart (DE)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: IdentXX GmbH, Stuttgart (DE)

(21) Appl. No.: 12/545,614

(22) Filed: Aug. 21, 2009

Related U.S. Application Data
(63) Continuation of application No. PCT/EP2008/001371, filed on Feb. 21, 2008.

(30) Foreign Application Priority Data
Feb. 23, 2007 (DE) DE 102007010311.7

(51) Int. Cl. C12Q 1/68

(52) U.S. Cl. 435/6; 435/91.2

(57) ABSTRACT

The present invention relates to a method for producing an organism-specific hybridizable nucleic acid molecule, a nucleic acid molecule produced by this method, a kit comprising at least any of the nucleic acid molecules, the use of a nucleotide sequence located between two elements on the genomic information of an organism for producing an organism-specific hybridizable nucleic acid molecule, and a method for detecting an organism in a biological sample.

### CROSS-REFERENCE TO RELATED APPLICATIONS

This application is a continuation of copending International Patent Application No. PCT/EP2008/001371, filed on Feb. 21, 2008, designating the United States, which was not published under PCT Article 21(2) in English, and in turn claims the priority of German Patent Application No. DE 10 2007 010 311, filed on Feb. 23, 2007. All these applications are incorporated herein by reference in their entirety.

### BACKGROUND OF THE INVENTION

1. Field of the Invention

The present invention relates to a method for producing an organism-specific hybridizable nucleic acid molecule, a nucleic acid molecule produced by this method, a kit which comprises at least one of said nucleic acid molecules, the use of a nucleotide sequence which is located between two elements on the genomic information of an organism, for producing an organism-specific hybridizable nucleic acid molecule, as well as a method for detecting an organism in a biological sample.

2. Related Prior Art

Hybridization refers to the combining of two single-stranded nucleic acids to double strands, which comprise complementary base sequences. A hybridization can occur between two DNA strands, one DNA and one RNA strand, as well as between two RNA strands.

Hybridizable nucleic acid molecules are e.g. primers for the polymerase chain reaction (PCR primers) or for other amplification methods which are based on the activity of a nucleic acid polymerase, and hybridization probes. The primers, e.g. for the PCR, or the hybridization probes can be DNA, RNA or synthetic oligonucleotide molecules, which can be used for the detection, the characterization or localization of such target nucleic acid molecules which comprise a nucleotide sequence which is complementary to that of the PCR primers or hybridization probes. Methods for producing PCR primers or hybridization probes are generally known in the art; cf. Sambrook and Russel (2001), Molecular Cloning—A Laboratory Handbook, 3. Edition, Cold Spring Harbor Laboratory Press, New York. The detection of the target nucleic acid molecule enables an indirect detection of the organism which carries the target nucleic acid molecule. By this manner e.g. pathogenic organisms can be detected, which contain the target nucleic acid molecule as a component of their genomic information. The identification of pathogenic organisms in turn is the prerequisite for a targeted therapy of a disease which is induced by that organism.

The modern phytopathology deals with diseases of plants caused by abiotic factors or biotic pathogens. Plant viruses, bacteria, fungi or parasitic flowering plants belong to these pathogens. The subject of the phytopathological research is e.g. the development of detection methods by means of which pathogens in seeds or plant material, soil samples, liquid substrates and any other media can be detected. Conventional test methods of the phytopathology are essentially limited to the planting of plants which possibly became ill and the visual characterization of the phenotype with regard to characteristic features of the disease (rating). However, these detection methods for plant diseases based on a visual rating are very time- and labor-intensive.

Alternatively, serologic methods for the detection of phytopathogens are employed where antibodies directed against the pathogens are used, e.g. within the context of a so-called “enzyme linked immunosorbent assay” (ELISA). However, serologic methods are very cost-intensive since the production of specific monoclonal antibodies requires extensive technical effort. Furthermore, due to the high homologies of the antigenic structures of eukaryotic pathogens it is often very difficult to develop highly specific antibodies against eukaryotes, which do not show a cross reactivity with closely-related species or with formae specialis, i.e. highly-specialized subspecies of a species.

With the detection methods which use the polymerase chain reaction (PCR) usually moderately variable genome segments of the pathogens, such as the so-called “internal transcribed spacer” (ITS), are amplified by the use of corresponding PCR primers. These moderately variable genome segments of the pathogens comprise very high sequence homologies across the species barrier. As a result of this, by this method species of pathogens which are closely related can hardly be distinguished. Furthermore, by means of these conventional PCR-based methods a differentiation of such pests within the same species, which infest or colonize different hosts or offormae specialis, respectively, is not possible.

The WO 95/33075 and DE 698 27 934 describe methods for the identification of the origin of DNA samples from DNA-containing organisms. For this purpose genetic fingerprints consisting of DNA amplificates of the DNA samples to be analyzed are established and compared to genetic fingerprints of DNA samples from reference organisms. However, this so-called “pattern matching” is disadvantageous. The patterns to be compared are in most cases not exactly identical what makes a comparison highly difficult. The effort is huge and it is not appropriate for a rapid identification as this is required for the agricultural practice.

### SUMMARY OF THE INVENTION

Against this background it is an object of the present invention to provide a method which enables the production of highly-selective, organism-specific, hybridizable nucleic acid molecules, which can be used within the context of a method for the detection of biotic pathogens, and by means of which the before-mentioned disadvantages of the art can be avoided. In particular, highly-selective, organism-specific nucleic acid molecules should be produced, which show a reduced cross reactivity with related pathogenic species or formae specialis.

This object is achieved by a method for producing an organism-specific hybridizable nucleic acid molecule, comprising the following steps: (1) providing a genomic information originating from an organism; (2) establishing a genetic fingerprint of said genomic information to obtain a pattern of amplificates of genome segments by the following steps: (2.1) amplifying segments of said genomic information of the organism to obtain a mixture of amplificates, and (2.2) separating the amplificates contained in the mixture to obtain a pattern of amplificates of genome segments; (3) isolating at least one amplificate from said pattern; (4) sequencing said isolated amplificate to obtain a nucleotide sequence; (5) selecting a segment of said nucleotide sequence comprising 3 to 1000 nucleotides, appropriate for the production of an organism-specific hybridizable nucleic acid molecule, and (6) producing a nucleic acid molecule comprising the segment selected in step (5).

According to the invention “organism” refers to any living being. In particular, it refers to living beings which can colonize or infest a vegetable, animal or human host. This may cause an illness of the host. In this connection it is particularly thought of plant diseases which are induced by plant viruses, bacteria, fungi, oomycetes, parasitic plants and animals etc.

According to the invention “genomic information” refers to a nucleic acid which is specific for the organism. In this context, in particular a genome in the form of DNA is meant, however also the RNA of the organism is meant, which results from a transcription of a part of the genome. According to the invention the genomic information comprises not only segments of the genome which carry information but also inter- and/or exogenic segments of the genome.

Methods for isolating genomic information from an organism are generally known in the art; Sambrook and Russell (loc. cit.); the content of this publication is hereby incorporated herein by reference.

To establish the genetic fingerprint in step 2 of the method according to the invention the following steps are performed: 2.1 amplifying segments of the genomic information of an organism to obtain a mixture of amplificates, and 2.2 separating the amplificates contained in the mixture to obtain a pattern of amplificates of genome segments.

“Genetic fingerprint” refers to characteristic genetic features of an organism, which enable its identification. This way of identification is based on the existence of specific nucleotide sequences in the genomic information of the organism. This applies for e.g. repetitive, tandem-like arranged DNA sequences. A prominent example of these repetitive DNA sequences relates to the so-called satellite DNA. Satellite DNA consists of a large number of copies of nucleotide sequences which are directly located one after the other. Examples of such short repeated sequences are A, AC, AAT or AAC, whereas also other sequences can be found. Satellite DNA with AC dinucleotide sequences can be averagely found once each 30 kb genome segment. Tri or tetranucleotide sequences are more seldom, once among several one hundred thousand base pairs. Further, in the genomic information of the organism longer repeating segments having a length of 5 to 2000 nucleotides can be found. These repetitive DNA sequences are distributed over the genome more or less statistically, whereas they accumulate at the centromeres and telomeres of the chromosomes.

The number of di, tri or tetranucleotides etc. in a satellite DNA is highly polymorphic. That means, two alleles differ with a high likelihood in the number of the di, tri or tetranucleotides of a given satellite DNA. The reason for these polymorphisms are displacements in DNA sequence repeats in the area of the replication forks. The satellite DNA is transmitted according to the Mendel rules.

Methods for generating genetic fingerprints which are used in step 2 according to the invention are comprehensively described in the art and are designated as “DNA finger-printing”, “DNA profiling” or “DNA typing”; they are e.g. described in Weising et al. (1995), “Fingerprinting in Plants and Fungi”, CRS Press, Boca Raton, Fla., USA; the content of this publication is incorporated herein by reference.

Accordingly, a large number of the repetitive segments, such as satellite DNA, has been sequenced in the meantime and methods for identifying further satellite DNA are comprehensively described in the art; cf. e.g. Kolpakov et al. (2003), “mreps: efficient and flexible detection of tandem repeats in DNA. Nucleic Acids Research 31 (13), pages 3672-3678. The content of this publication is incorporated herein by reference. The computer program “mreps” which enables the identification of satellite DNA can e.g. be obtained on the website “Bioinfo” of the bioinformatic group at the “Universite des Sciences et Technologies de Lille”, France, or on the website of the “Bioinformation Systems e.V., Verein zur Förderung der Datenverwertung in der molekularen Biologie”. Further computer programs for identifying satellite DNA are “SciRoKo”, “MISA”, “SSRFinder”, “SSRIT”, “TRF”, “Sputnik”.

The nucleotide sequences of numerous satellite DNAs can be obtained from data bases which are accessible to the public. Satellite DNA from plants, animals and fungi are e.g. disclosed on the website of the “International Crops Research Institute for the Semi-Arid Tropics (CRISAT)”. Satellite DNAs from plants are also disclosed on the website “PlantSat” of the Laboratory for molecular cytogenetic, Institut for Molecular Plant Biology, Ceske Budejovice, Czech Republic. The content of these data bases or websites, respectively, is incorporated herein by reference.

Based on these sequence information PCR primers can be produced which hybridize to the satellite DNA. It goes without saying that the forward and backward primer can each hybridize either to the same satellite DNA but also to different satellite DNAs i.e. different nucleotide sequences. It is only important that the segments which are located between such repetitive segments of the genomic information, are amplified, which are in the following referred to as “amplificates”. In the given example this occurs by means of a PCR reaction. It goes without saying that further methods for the amplification of segments of nucleic acids can be used, such as the amplification by means of phi-DNA-polymerases or the thermophilic helicase-dependent amplification (tHDA).

According to the invention, the corresponding amplificates can be separated, e.g. according to their size, by means of chromatographic or electrophoretic methods and can be visualized via colorants which specifically bind to or associate with the DNA. This results in a pattern of amplificates, which is, due to the mentioned polymorphism, characteristical for each organism to be analyzed. This pattern is designated as genetic fingerprint. In an extreme example where the primer or primers, respectively, which are used for DNA fingerprinting, amplify only one single fragment of the genomic information, the pattern according to the invention can consist of one amplificate only. It is to be understood according to the invention that “one” amplificate means the existence of several nucleic acid molecules having the identical nucleotide sequence, which, as a consequence, do not differ from each other.

In the next step 3 according to the invention “one” amplificate is isolated from the genetic fingerprint or pattern of amplificates, respectively. This can be e.g. realized by excising one band which contains the nucleic acid of “one” amplificate from the electrophoresis gel by means of a cutter, and by eluting the nucleic acid contained therein from the gel according to generally known methods.

The sequenzing of the isolated amplificate in the next step according to the invention is also performed according to methods which are generally known in the art, e.g. by means of the Maxam Gilbert or the Sanger method; cf. Sambrook and Russell (loc. cit.).

After the nucleotide sequence of the isolated amplificate is obtained in step 5 such a segment is selected which is suitable for producing a hybridizable nucleic acid molecule. This segment comprises 3 to 1000 nucleotides. The selection criteria which are used in this context are generally known to the skilled person. If e.g. the production of a PCR primer is intended preferably segments are selected which comprise a length of 3 or 10 to 36 nucleotides, preferably 18 to 25 nucleotides, which can however be shorter or longer. The criteria considered by the skilled person for producing a PCR primer are e.g. disclosed in Sambrook and Russel (loc. cit.), chapter 8, table 8.3, the content of this table is incorporated herein by reference. The selection criteria can be summarized as follows:
The G+C content should be between 40% and 60%, preferably between 45% and 55%, with a regular distribution of all four nucleotides along the length of the primer.
To increase the amplification capability it is advantageous if the primer comprises at the 3′ end the nucleotide G or C. It is ideal if the last two nucleotides at the 3′ end comprise the variations GG; GC; CG or CC.
The PCR primer should not hybridize to itself. It is ideal if ≦3 identical nucleotides are consecutively arranged.
When selecting the forward and backward primers care is to be taken that they cannot hybridize with each other. It is ideal if both primers comprise ≦3 complementary nucleotides. The distance of both primers should be chosen in such a manner that the amplificate can be properly detected in the detection system used in each case. Optimum for polyacrylamide gels: 100-1500 nucleotides; optimum for agarose gels: 250-750 nucleotides; optimum for real-time PCR systems: 100-250 nucleotides.
To enable a specific detection the primer should be constructed such that a hybridization temperature of ≧50° C. is possible. The hybridization temperatures applicable for a primer can be calculated by specifically provided software.

The actual production of the species-specific hybridizable nucleic acid molecule occurs by the use of standard methods of the nucleic acid synthesis which are known to the skilled person; cf. also Sambrook and Russell (loc. cit.).

The object underlying the invention is herewith completely achieved.

The inventors have realized that amplificates, which are obtained when establishing a genetic fingerprint of an organism, are suitable matrices for the production of highly selective, organism-specific and hybridizable nucleic acid molecules, such as probes and PCR primers. The inventors have realized that within the context of the DNA fingerprinting fragments of the genomic information of the interesting organism can be amplified, which are usually arranged between the genes of the organism, i.e. intergenically, i.e. which do not contain information encoding a protein. Due to the low or even lacking selection pressure these fragments comprise a very high variability between individual organisms and, therefore, a high specificity for the particular organism. By means of the method according to the invention nucleic acid molecules can be obtained which can bind to such fragments in a highly-selective manner. Examples of such nucleic acid molecules are probes and PCR primers or DNA oligomeres, respectively. By using methods known in the art a skilled person, such as a molecular biologist or a plant pathologist, can determine whether such a nucleic acid molecule produced according to the invention, can hybridize to genomic information in any biological sample. In the case of a hybridization the conclusion can be drawn that the organism searched for is contained in the sample.

It is of a particular advantage that by means of the PCR primers or hybridization probes, respectively, which were produced via the method according to the invention, it cannot only be distinguished between different species but also between individuals in a highly-selective manner. This also enables the detection of formae specialis or of pathogenic subspecies within one species, respectively, and the conditions are established for a targeted treatment of an infested plant.

It is preferred for the method according to the invention if the genomic information of the organism is double-stranded DNA.

This measure has the advantage that the genomic, double-stranded DNA of the organism is particularity suitable for establishing a genetic fingerprint. In the art numerous methods are described which enable establishing the genetic fingerprint on the basis of double-stranded DNA; cf. Weising et al. (loc. cit.). These comprise the PCR-based method of DNA fingerprinting which is explained in detail at the outset.

The amplification in step (2.1) is preferably realized by means of a polymerase chain reaction (PCR), whereas in particular segments of a satellite DNA, in particular of a microsatellite DNA and simple sequence repeats (SSRs), are suitable as primer annealing sides.

“Satellite DNA” refers to frequently repeated, i.e. repetitive DNA sequences, which can be mainly found in the genome of eukaryotes, and which can, according to the organism, constitute up to 90% of the total genome. Satellite DNAs are named differently according to the length of their repetitive nucleotide sequence and the number of these tandem-like arranged copies. Smaller satellite DNA is often referred to as “microsatellite DNA”. This is to be understood as being di, tri or tetranucleotides of which 10 to 30 copies can be found within one “clustered” repetition unit. Microsatellite DNA also comprise repetitive sequences of 20 to 40 nucleotides and extends over an area of several kilobases. They are also referred to as “variable number of tandem repeats” (VNTR). “Single sequence repeats” (SSRs) which are also designated as “simple sequence repeats” are short, repetitive sequences comprising 1 to 4 nucleotides.

This measure has the advantage that the nucleotide sequences of a large number of satellite DNAs are described in the art. It is, therefore, an easy measure for a person skilled in the art to generate suitable PCR primers which hybridize to satellite DNA. Nucleotide sequences of satellite DNAs can e.g. be found on the website of the “International Crops Research Institute for the Semi-Arid Tropics (ICRISAT)” (loc. cit.). The content of this website is incorporated herein by reference. Satellite DNA can be generally represented as (Nx)n, whereas N is any nucleotide, and x is an integer from 2-6 for SSRs, x is an integer from 7-30 for microsatellite DNA, and x is an integer from 31-10000 for macrosatellite DNA; n is generally an integer from 2 to 1000, whereas larger values might be possible for both n as well as for x.

In the method according to the invention for the PCR alternatively such a forward primer can be selected which binds to a randomized nucleotide sequence on the first DNA strand of the organism comprising a length of 3 to 30, preferably of 4 to 20, highly preferred of 6 to 10 nucleotides, and such a backward primer which hybridizes to a nucleotide sequence on the DNA strand complementary to the first DNA strand, comprising a length of 3 to 30, preferably of 4 to 20, highly preferred of 6 to 10 nucleotides.

Also this measure enables the establishing of the genetic fingerprint in form of a pattern of amplificates of fragments of the genomic information of the interesting organism. For this purpose a forward PCR primer is generated which comprises 3 to 10 arbitrarily selected nucleotides. The backward PCR primer is selected correspondingly. The inventors have realized that, for statistic reasons, in a large number of organisms corresponding nucleotide sequences can be found as being spread over the genome. Within the context of the PCR reaction the fragments of the genomic information of the organism, which are arranged in-between, are amplified, which comprise for statistic reasons, according to the findings of the inventors, also a very high organism specificity, since a large part of the genome does not comprise information and is, therefore, suitable for the production of organism-specific hybridizable nucleic acid molecules.

In the method according to the invention within the context of the PCR alternatively such a forward primer is selected which hybridizes to a nucleotide sequence on the first DNA strand of the organism, which is ascribable to a virus genome, and such a backward primer is selected which can hybridize to such a nucleotide sequence on the DNA strand complementary to the first DNA strand, which is ascribable to a virus genome.

Also by this measure amplificates of such genome fragments can be obtained within the context of a DNA fingerprinting, which enable the production of species-specific hybridizable nucleic acid molecules. Nucleotide sequences which are ascribable to a virus genome can be identified on the basis of sequence homologies. According to the invention, it is referred to a “nucleotide sequence ascribable to a virus genome” if segments of the genomic information of at least 10 nucleotides comprise a homology with a corresponding segment of a virus genome of at least 30%, preferably of at least 40%, 50%, 60%, 70%, 80%, 90%, 95% and highly preferred of at least 100%. The homologies can be determined e.g. by means of the software MegAlign of the laser gene program of DNAStar Inc.

Nucleotide sequences ascribable to a virus genome are comprehensively described in the art, cf. Cooper and Geoffrey (2000), “The Cell—A molecular approach”, 2. edition, Sunderland (MA): Sinauer Associates, Inc.; Brown, T. A. (2002), “Genomes” 2. edition, New York and London: Garland Science; Pearson and Morrow (1981), “Discrete-length repeated sequences in eukaryotic genomes”, Proceedings of the National Academy of Sciences of the United States of America, vol. 78, pages 4016-4020; Bernardi, G. (2005), “Structural and evolutionary genomics—natural selection in genome evolution”, Elsevier Science Publications, and Baltimore, D. (1985), “Retroviruses and retrotransposons: the role of reverse transcription in shaping the eukaryotic genome”, Cell, 40 (3), pages 481-482. In the sequence listing attached to this application under SEQ ID No. 37 an example of a sequence ascribable to a virus genome is given, from which suitable PCR primers can be deduced. This sequence originates from the publication of Shull and Hamer (1996), “Genetic differentiation in the rice blast fungus revealed by the distribution of the Fosbury retrotransposon”, Fungal Genetics and Biology 20 (1), pages 59-69. Further examples of such sequences can be found on the “Medline” website of the “National Center for Biotechnology information” (NCBI) under the keyword LTR. The content of the before-mentioned publication and the website is incorporated herein by reference.

In the method according to the invention within the context of the PCR alternatively such a forward primer is selected which hybridizes to a nucleotide sequence of a transposable element on the first DNA strand of the organism, and such a backward primer is selected which hybridizes to a nucleotide sequence of a transposable element on such a DNA strand of the organism complementary to the first strand.

Transposable or transponable elements are mobile genetic elements, i.e. areas of DNA which can change their position within the genome. The inventors have realized that such transposable elements are spread over the genome similar to e.g. satellite DNA, and genome segments which are arranged in-between are mostly intergenic and highly variable between organisms and individuals. The art also describes transposable elements which simultaneously are sequences ascribable to a virus genome; cf. Cooper and Geoffrey (loc. cit.); Brown, T. A. (loc. cit.); Pearson and Morrow (loc. cit.); Bernardi, G. (loc. cit.) and Baltimore, D. (loc. cit.).

In the method according to the invention within the context of the PCR such a forward primer is selected which hybridizes to a repetitive nucleotide sequence of a telomere or centromere on the first DNA strand of the organism, and such a backward primer is selected which hybridizes to a repetitive nucleotide sequence of a telomere or centromere on a DNA strand of the organism complementary to the first DNA strand.

According to the inventors, also by this measure a genetic fingerprint of the genetic information of the organism in form of a pattern of amplificates of genome fragments can be established. It is known that in the regions of the centromeres or telomeres of chromosomes repeats of hundreds or thousands of DNA segments connected in series are arranged, the organization of which corresponds to that of satellite DNA. These repetitive nucleotide sequences do also comprise a very high variability between individuals and are, therefore, also suitable for developing highly-specific PCR probes and primers for the detection of these individuals or organisms, respectively. Repetitive nucleotide sequences in the regions of centromeres and telomeres of chromosomes are comprehensively described in the art; cf. Brown, T. A. (loc. cit.); Griffiths, et al. (1999), “Modern genetic analysis”, New York: W.H. Freeman & Co.; Cooper and Geoffrey (loc. cit.) and Alberts et al. (2002), “Molecular biology of the cell” 4. edition, New York and London: Garland Science. In the attached sequence listing an example of such a sequence is given under SEQ ID no. 38, which originates from Borovkov and McClean (1993), “A tandemly repeated sequence from the Plasmopara halstedii genome”, genes 124 (1), pages 127-130, and which has already been successfully used by the inventors to produce an organism-specific hybridizable nucleic acid molecule, which is suitable to detect Plasmopara halstedii with high sensitivity and specificity (see also the embodiment). The before-identified publications are incorporated herein by reference.

The inventors have realized that fragments of a genomic information which can be obtained by means of a DNA fingerprinting of organisms, comprise a very high variability not only between organisms of different species but also between form a specialis and even individuals and, therefore, are particularity suitable for the production of organism-specific hybridizable nucleic acid molecules, e.g. in form of PCR primers and/or hybridization probes. These fragments are usually located between repetitive DNA sequences which are statistically spread over the genome of the microorganism, such as between satellite DNA or also between nucleotide sequences ascribable to a virus genome, between transposable elements or between repetitive nucleotide sequences of a telomere or centromere. Also nucleotide sequences, which are located between randomized short nucleotide sequences of 3 to 30 nucleotides can be used for this purpose.

Against this background the present invention also relates to the use of a nucleic acid molecule comprising a nucleotide sequence which is located between two elements on the genetic information of an organism for producing an organism-specific hybridizable nucleic acid molecule, preferably a PCR primer, whereas the two elements are each selected, independingly from each other, from the group consisting of: satellite DNA nucleotide sequence, nucleotide sequence ascribable to a virus genome, nucleotide sequence of a transposable element, repetitive nucleotide sequence of a telomere/centromere.

According to the invention “between two elements on the genetic information of a organism” means that such nucleotide sequence is flanked at its 5′ terminus and 3′ terminus by the two elements, i.e. the one element borders to the 5′ end of the nucleotide sequence, and the other element borders to the 3′ terminus.

Examples of nucleotide sequences of such nucleic acid molecules are given in the attached sequence listing. These nucleic acid molecules are derived from the genomes of the plant pathogens Peronospora arborescens, Peronospora swinglei s.l., Fusarium oxysporuni, Plasmopara petroselini and Peronospora valerianellae and are flanked at their 5′ terminus and their 3′ terminus by microsatellites or single sequence repeats (SSRs). These exemplary nucleotide sequences are listed in the attached sequence listing under SEQ ID No. 1 to 8; cf. also table 1.

Against this background another subject-matter of the present invention is a nucleic acid molecule which comprises any of the nucleotide sequences of SEQ ID No. 1 to 8 of the enclosed sequence listing or a nucleotide sequence complementary thereto.

The mentioned nucleic acid molecules turned out to be particularly suitable to function as a matrix for the production of hybridisable nucleic acid molecules by which the organisms Peronospora arborescens, Peronospora swinglei s.l., Fusarium oxysporum, Plasmopara petroselini and Peronospora valerianellae can be detected in a biological sample.

It is preferred according to the invention if the before-identified nucleic acid molecules comprise a nucleotide sequence of 3 to 812, preferably of 10 to 64, further preferred of 12 to 48, further preferred of 14 to 36, further preferred of 16 to 27 consecutively arranged nucleotides of any of the nucleotide sequences of SEQ ID No. 1 to 8 or a nucleotide sequence complementary thereto.

By this measure nucleic acid molecules are already provided which comprise a segment with a length sufficient to function as a PCR primer for the detection of the organisms Peronosporci arborescens, Peronospora swinglei s.l., Fusarium oxysporum, Plasmopara petroselini and Peronospora valerianellae. These PCR primers are capable to hybridize to such nucleic acid molecules which comprise the nucleotide sequences of SEQ ID No. 1 to 8 or a sequence complementary thereto.

Another subject-matter of the present invention relates to a nucleic acid molecule which comprises at least 20%, preferably at least 30%, further preferred 40%, further preferred 50%, further preferred 60%, further preferred 70%, further preferred 80%, further preferred 90%, further preferred 95%, further preferred 99% sequence homology to the before-identified nucleic acid molecules.

Due to the high homology also with such nucleic acid molecules the three mentioned plant pathogenic microorganism can be detected in a highly selective and specific manner. The homologies can be easily identified by means of methods known to the skilled person, e.g. a BLAST analysis or by means of the MegAlign module of the laser gene program of DNAStar Inc. The indicated identities refer to the entire before-identified nucleic acid molecule according to the invention or to a segment thereof having ≧10 nucleotides, respectively.

Another subject-matter relates to such a nucleic acid molecule which, under stringent conditions, hybridizes to any of the before-identified nucleic acid molecules.

Such a nucleic acid molecule also comprises the properties of the before-described nucleic acid molecules and is, therefore, suitable for specifically detecting the plant pathogens. “Stringent conditions” refers to such conditions under which only such nucleic acid strands can form base pairs which are nearly perfectly complementary, i.e. at 80%, 85%, 90%, 95%, 98%, 99%. Stringent conditions can be adjusted by changing the salt concentrations, the pH value, the temperature etc. in the medium where the binding of the nucleic acid strands should take place.

Other criteria which are to be considered by the skilled person when designing the PCR primers can be found further above in connection with the explanation of the step 5 of the method according to the invention and also in Sambrook and Russel (loc. cit.), chapter 8, table 8.3, the content of this table is hereby incorporated herein by reference.

Another subject-matter of the invention relates to a method for producing an organism-specific hybridizable nucleic acid molecule, comprising the following steps: (1) providing a genomic information originating from an organism; (2) incubating said genomic information of the organism with at least one restriction endonuclease to obtain a mixture of restriction fragments; (3) ligating oligonucleotides of a defined nucleotide sequence (adaptor sequence) to the free ends of the restriction fragments to obtain modified segments of the genomic information; (4) establishing a genetic fingerprint of the genomic information to obtain a pattern of amplificates of genome segments by the following steps: (4.1) amplifying said fragments of the genomic information of the organism to obtain a mixture of amplificates, (4.2) separating said amplificates contained in the mixture to obtain a pattern of amplificates of genome segments; (5) isolating at least one amplificate from the pattern; (6) sequencing said isolated amplificate to obtain a nucleotide sequence; (7) selecting a segment of said nucleotide sequence of 3 to 1000 nucleotides suitable for producing an organism-specific hybridizable nucleic acid molecule, and (8) producing a nucleic acid molecule comprising said segment selected in step (6), whereby the amplification in step 4.1 is performed by means of a polymerase chain reaction (PCR), and such a forward primer is selected which hybridizes to the adaptor sequence on the first DNA strand of the organism, and such a backward PCR primer is selected which hybridizes to the adaptor sequence of a DNA strand of the organism complementary to the first DNA strand.

With this method within the context of the DNA fingerprinting an established method designated as “amplified fragment length polymorphism” (AFLP) is used which also results in a pattern of amplificates of genome fragments having the characteristics which are required according to the invention. In the first step the genomic information of the interesting organism is cleaved by a restriction endonuclease having a known specificity for a cleaving side. In the next step short nucleic acid molecules are provided as so-called adaptors which are designed by the skilled person in such a way that they are complementary to the ends of the generated restriction fragments and also comprise hybridization sides for PCR primers for the following PCR step. Therefore, the adaptors comprise a sequence known to the skilled person, which is oriented by the restriction cleavage side and the sequence of the PCR primers for the following amplification. In the next step an amplification of the ligation products or the genomic fragments, respectively, is performed by means of a polymerase chain reaction (PCR). For this purpose primers are used which fit to the adaptors. The genome fragment located between the adaptors is obtained in form of the amplificate. Optionally measures for reducing the number of genomic fragments can be taken, in case such number is particularity high due to the selected restriction endonuclease and, therefore, is poorly manageable. For this purpose e.g. additional amplifications can follow which results in a reduction of the genomic fragments. The AFLP method is e.g. described in Weising et al. (loc. cit.). This publication is hereby incorporated herein by reference.

Another subject-matter of the invention relates to a method for producing an organism-specific hybridizable nucleic acid molecule, comprising the following steps: (1) providing a genomic information originating from an organism; (2) establishing a genetic fingerprint of the genomic information to obtain a pattern of amplificates of genome segments by the following steps: (2.1) incubating the genomic information with at least one restriction endonuclease to obtain a mixture of fragments of the genomic information, (2.2) ligating the fragments of the genomic information into vectors, (2.3) introducing the vectors into host organisms, optionally (2.3°) plating the host organisms onto cultivation plates, (2.4) amplifying the vectors in the host organisms, (2.5) isolating the amplified vectors to obtain a mixture of amplificates of genome segments, and (2.6) separating said amplificates contained in the mixture to obtain a pattern of amplificates of genome segments; (3) isolating at least one amplificate from the pattern; (4) sequencing said isolated amplificate to obtain a nucleotide sequence; (5) selecting a segment of said nucleotide sequence of 3 to 1000 nucleotides, suitable for producing an organism-specific hybridizable nucleic acid molecule, and (6) producing a nucleic acid molecule comprising said segment selected in step (5).

According to the findings of the inventors, this method represents an alternative proceedings to establish a genetic fingerprint, which can be performed by reagents and methods which are customary in the laboratory.

In the method according to the invention the separation of the amplificates contained in the mixture is e.g. realized by means of a chromatographic and/or electrophoretic method.

Particularly well-suited are electrophoretic methods where the amplificates are separated from each other according to their size by migrating in an electrical field. Also suitable are, however, chromatographic methods, such as the high performance liquid chromatography (HPLC) as well as all methods which are suitable to separate nucleic acid molecules of different composition and/or length and/or charge contained in a mixture. Such methods are generally known to the skilled person; cf. Sambrook and Russell (loc. cit.).

In the method according to the invention it is preferred if at least one amplificate is isolated from the pattern of amplificates of genome segments, which is, in relation to the remaining amplificates, more amplified than average.

The inventors have realized that such amplificates which prevail in a high quantity are particularly well-suited as a matrix for producing a species-specific hybridizable nucleic acid molecule. The segments such amplificates are based on, exist in the genomic information or the genome of the interesting organism either in a high number of copies or do barely form interfering secondary structures in the amplification. Such segments which can particularily well be amplified can, e.g. after a gel electrophoretic separation of the mixture of amplificates and a subsequent staining with an appropriate substance, such as ethidiumbromide, be detected due to a particularity strong or prominent gel band. The gel bands e.g. can be densidometrically measured and an average of the measured data can be formed. An amplification of an amplificate is “higher than average” if in the densidometry the measured data beyond average can be referred to a gel band. It is particularity preferred if an amplificate which is amplified higher than average is two times, three times, four times, five times or ten times stronger amplified than the average of the amplificates. The isolation of such an amplificate which is higher amplified than average is e.g. realized by excising the corresponding band from the gel and eluting the amplificate contained therein by means of methods known in the art.

It is also preferred in the methods according to the invention if, after the isolation of at least one amplificate from the pattern and before the sequencing of the isolated amplificate, the following further steps are performed: a) inserting the isolated amplificate into a vector, b) introducing the vector into an appropriate organism, and c) isolating the vector from the organism to obtain at least one isolated amplificate.

By these measures starting points for the subsequent sequencing are created. The organisms, preferably microorganisms such as Escherichia coli, “repair” the vector and make the amplificate particularity well sequenzable. The amplificate contained in the vector is therewith provided in ideal form for the subsequent method steps.

The segment selected for producing an organism-specific hybridizable nucleic acid molecule preferably comprises a length of 10 to 64, further preferred 12 to 48, further preferred 14 to 36, further preferred 16 to 27 nucleotides, whereas it is further preferred to provide the nucleic acid molecule produced in the subsequent step as a PCR primer or, alternatively, as a hybridization probe.

According to the experience hybridization probes or PCR primers, respectively, comprise the before-identified number of nucleotides, which are in their sequence complementary to the target sequence to be detected. Hybridization probes and PCR primers can comprise a length of 5, 10, 15, 20, 30, 50, 100, 150 or 200 to 2000 nucleotides, respectively, and their sequence is complementary to that of the target sequence. Ideally the number of nucleotides is 20±10. Criteria considered by the skilled person when designing PCR primers can be found in Sambrook and Russel (loc. cit.), chapter 8, table 8.3, the content of this table is hereby incorporated herein by reference.

The inventors have already produced a large number of organism-specific hybridizable nucleic acid molecules which can be used as PCR primers and are identified in the attached sequence listing under SEQ ID No. 9 to 35; cf. also table 2.

The subject-matter of the present invention is, therefore, also a nucleic acid molecule comprising one of the nucleotide sequences of SEQ ID No. 9 to 35 of the attached sequence listing or a nucleotide sequence complementary thereto.

Another subject-matter of the present invention relates to a nucleic acid molecule which comprises at least 20%, preferably at least 30%, further preferred 40%, further preferred 50%, further preferred 60%, further preferred 70%, further preferred 80%, further preferred 90%, further preferred 95%, further preferred 99% sequence homology with the before-identified nucleic acid molecules.

Due to the high homology also with such nucleic acid molecules a plant pathogenic microorganism can be detected in a highly selective and species-specific manner. The homologies can be easily identified by means of methods known to the skilled person, e.g. a BLAST analysis or by means of the MegAlign module of the laser gene program of DNAStar Inc. The indicated identities relate to the entire before-identified nucleic acid molecules according to the invention or to a segment thereof with ≧10 nucleotides.

Another subject-matter of the present invention relates to a nucleic acid molecule which, under stringent conditions, hybridizes to one of the before-described nucleic acid molecules.

Also such a nucleic acid molecule comprises the characteristics of the before-described nucleic acid molecules and is, therefore, suitable for a specific detection of the mentioned microorganisms. With regard to the determination of stringent conditions it is referred to the definition given further above.

Another subject-matter of the present invention relates to a kit which comprises at least one of the before-identified nucleic acid molecules, a manual for using said nucleic acid molecule, preferably within the context of the following detection method according to the invention, and, if appropriate, buffer and chemicals.

Such kits can e.g. be assembled as diagnosis kit for any microorganism to be detected. Such a diagnosis kit then contains e.g. the PCR primer which is highly selective for the relevant pathogenic organism to be detected, and a DNA sample of the corresponding organism as a positive control. Further, such a diagnosis kit contains a manual with detailed information on how to perform the subsequent detection method. It is to be understood that the kit can contain appropriate buffers, chemicals and other auxiliary substances. The provision of such akit has the advantage that all required chemicals and information are provided, which also enables a corresponding detection method to be performed by low-skilled labor without mistakes, if applicable.

Another subject-matter of the invention relates to a method for detecting an organism in a biological sample, e.g. a seed sample, comprising the following steps: 1. providing a biological sample containing a nucleic acid, 2. isolating said nucleic acid from the sample, 3. subjecting said isolated nucleic acid to a polymerase chain reaction (PCR), 4. detecting whether in step 3 an amplification of said isolated nucleic acid has taken place, and 5. correlating a positive determination in step 4 with the presence of the organism in the sample, and correlating a negative determination in step 4 with the absence of the organism in the sample, whereas in step 3 at least one nucleic acid molecule is used as a PCR primer, which has been produced by the method according to the invention.

For the before-described method the explanations given above in connection with the remaining subject-matter according to the invention apply correspondingly. It is explicitly referred thereto.

The PCR reaction is preferably performed as “nested” or interlaced PCR reaction.

This measure has the particular advantage that the specificity and efficiency of the performed PCR is further increased. For this in a first PCR round within a so-called pre PCR a first template is amplified in few cycles. Here the primers are selected in such a manner that they have a relative large distance to each other, i.e. the forward PCR primer hybridizes e.g. relatively close to the 3′ end, whereas the anti-sense PCR primer hybridizes e.g. closely to the 5′ end of the intergenic organism-specific DNA segment. The amplificate of this pre PCR is then amplified by a further PCR round, the secondary PCR or host PCR by the use of a new PCR primer pair. The new PCR primers are more located in the interior in comparison to the first primers so that in this second step only the specific DNA segments from the pre PCR are amplified. Consequently, the sensitivity and efficiency of the method according to the invention is markably increased.

It is to be understood that the features mentioned before and those to be explained in the following cannot only be used in the indicated combination but also in other combinations or alone, without departing the scope of the present invention.

In the following embodiments of the invention are explained in more detail resulting in further characteristics and features. However, the embodiments do not limit the scope of the invention. Reference is made to the enclosed figures which show the following:

### BRIEF DESCRIPTION OF THE DRAWINGS

FIG. 1 shows a conventional DNA fingerprint for performing a pattern matching according to the art, to identify the pathogen Xatnthomonas campestris in field samples of kohlrabi;

FIG. 2 shows a schematic presentation of the occurance of organism-specific DNA sequences with flanking repetitive DNA sequences in the example of an eukaryotic chromosome;

FIG. 3 shows a schematic presentation of important steps of the method according to the invention for producing organism-specific nucleic acid molecules;

FIG. 4 shows the result of the detection method according to the invention on the basis of an agarose gel stained by ethidiumbromide, to detect the pathogen Peronospora swinglei s.l. in seeds of basil.

### DETAILED DESCRIPTION OF EMBODIMENTS

### Example 1

### Detection of Xanthomonas campestris via a “Pattern Matching” (Prior Art)

The bacterial pathogen Xanthomonas campestris pathovar campestris is one of the most important pathogens in the cultivation of cabbage. For the identification of the organism in the art a method is used which is based on the comparison of genetic fingerprints (“pattern matching”). The method and the problems involved should be demonstrated in the following by means of 9 pure cultures isolated from the field cultivation of kohlrabi and different wild plants.

The standard protocol in the art comprises essentially the following 4 working steps, namely (a) the provision of organisms in form of a pure culture, (b) the DNA extraction, (c) the production of a genetic fingerprint, and (d) the electrophoretic separation of the genetic fingerprints.

### (a) Provision of the Organisms in Form of a Pure Culture

The parts of the plants which have been macroscopically identified as being affected by the disease were transferred into a sterile test tube which contained liquid medium, such as the NYG medium. After an incubation period of at least 12 hours at 30° C. dilution smears were prepared on agar plates comprising solutions of the liquid medium of different concentrations, and were incubated at 30° C. over night. At the other day individual colonies of bacteria were removed from the agar plate and further dilution smears were prepared. This process was as often repeated as it became certain that the smear was in fact a pure culture of the organism. These pure cultures were used according to the Koch's postulates in re-infections to verify the pathogenicity. For the further experiments only such pure cultures have been used which showed the typical symptoms of infection by the pathogen.

### (b) The DNA Extraction

The extraction of the bacterial DNA was performed with the GenElute™ bacterial genomic DNA kit (Sigma Aldrich, Deutschland) according to the information of the manufacture. In the following the obtained DNA solution was photometrically tested in view of quantity and quality.

### (c) Establishing a Genetic Fingerprint

A PCR reaction is prepared in a reaction vial which is adapted to a PCR, e.g. consisting of 2.5 μl 10×PCR buffer, 2.5 μl of a 2 mM solution of the four deoxynucleotide triphosphates (dNTPs), 2.5 μl magnesium chloride solution (20 mM), 1 U of a thermostable DNA polymerase, e.g. Taq DNA polymerase, 1 μl of an e.g. 25 mM solution of the PCR primer GTC₅ (5′GTCGTCGTCGTCGTC-3′; SEQ ID no. 39). The primer is designed in such a manner that it can bind to the microsatellite GTC. The sample is filled up to 25 μl with water. In the following, the PCR is performed e.g. under the following conditions: The incubation in a thermocycler is performed according to the specific modalities of the apparatus. At first e.g. an incubation is performed, e.g. at 94° C. for 2 minutes. In the following an incubation at e.g. 94° C. is performed for 1 minute. In the following an incubation, e.g. at 56° C. for 1 minute. Subsequently, an incubation at e.g. 72° C. for 2 minutes is performed. In the following the last three steps are repeated, e.g. 30 times. Then e.g. a final incubation can be performed, e.g. at 72° C. for 4 minutes.

### (d) Electrophoretic Separation of the Genetic Fingerprints

The amplificates which were obtained in the PCR are separated, e.g. by means of gel electrophoresis, e.g. in an agarose gel. This can e.g. be prepared in putting 1 g agarose into a receptacle, filled up to 100 μl with an ion-containing liquid, e.g. with Tris-borat-EDTA buffer (TBE).

In the following the PCR reaction solution is put into a pre-form cavity of the agarose gel, e.g. after the mixture with ⅙ vol. glycerin and colorants, if applicable, which migrate in the electrophoresis in the same direction like the DNA fragments in the agarose gel and to control the progress of the electrophoresis. The electrophoresis itself is performed in an adapted electrophoresis chamber which is filled with an ion-containing solution which is isotonic to the gel, preferably with the same solution which was used for the preparation of the agarose gel. In applying a voltage, e.g. 140 V, the migration of the DNA fragments through the agarose gel is forced, which are separated on account of the selective retention which is in particular based on the size of the migrating fragments. In the following a staining is performed by a colorant which associates with or binds to nucleic acids, e.g. ethidiumbromide, e.g. by the incubation of the agarose gel for half an hour in an ethidiumbromide-containing staining bath.

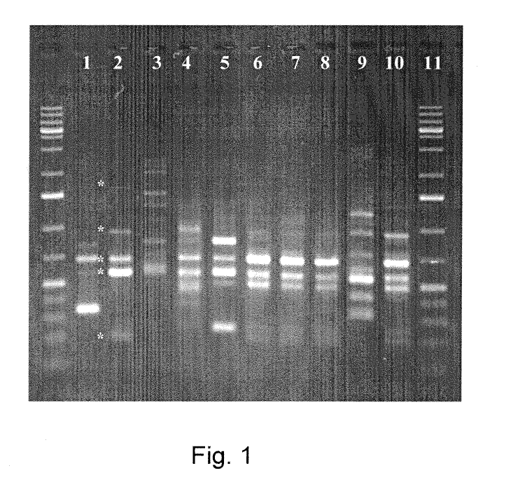

The result of this experiment is shown in FIG. 1: agarose gel, 1.5%, stained with ethidiumbromide; used primers: GTC₅; lane 1: Xanthomonas campestris field sample 1; lane 2: Xanthomonas campestris reference sample; lane 3: Xanthomonas campestris field sample 2; lane 4: Xanthomonas campestris field sample 3; lane 5: Xanthomonas campestris field sample 4; lane 6: Xanthomonas campestris field sample 5; lane 7: Xanthomonas campestris field sample 6; lane 8: Xanthomonas campestris field sample 7; lane 9: Xanthomonas campestris field sample 8; lane 10: Xanthomonas campestris field sample 9; lane 11: DNA size marker (standard) 1.

By means of the used primers GTC₅ for the reference sample of Xanthomonas campestris pathovar campestris (lane 2) five distinct amplificates with different sizes could be obtained (labeled with *). If this reference pattern is compared to the other nine patterns of the organisms to be identified, it can be realized that none of these nine patterns is 100% identical with the reference pattern. With this examination a reliable identification of the organisms cannot be made.

The presented example shows the typical disadvantages of the identification on the basis of a “pattern matching” of genetic fingerprints. The patterns to be compared are in most cases not exactly identical, some amplificates are weaker or stronger, amplificates lack completely or new ones appear. To minimize these problems in practice different fingerprints, based on different PCR primers, are established with the DNA solution. Each of the comparison analyses is then compared to each other in a complex procedure. The complexity is big and it is not appropriate for a rapid identification which is required for the agricultural practice. In addition, the result very strongly depends on the used separation matrix or the conditions of separation in general, respectively, which makes the comparability of the test results of the individual laboratories very difficult.

### Example 2

### Organism-Specific DNA Regions with Flanking Repetitive DNA Sequences

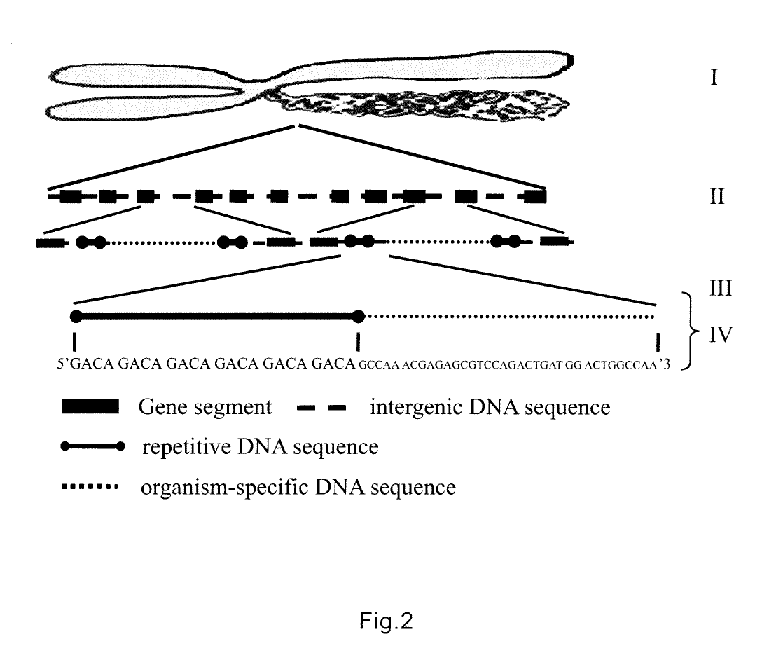

FIG. 2 shows schematically a part of the genome of a phytopathogen. In the organization level I a chromosome is schematically shown; the organization level II shows the DNA double strand on which the encoding gene segments and regions of intergenic DNA sequences are shown; the organization level IV shows a sequence example for repetitive (e.g. microsatellites; continuous line) and organism-specific (dotted line) DNA sequences.

The intergenic sequences including organism-specific DNA sequences which are located between repetitive sequences or which are flanked by these sequences, are excellently suited as matrices for the production of organism-specific hybridizable nucleic acid molecules, such as PCR primers, namely for the following reasons: intergenic sequences are not encoding proteins (FIG. 2 (II)), which results in a low or lacking selection pressure. They have a very high variability between individual organisms or species, respectively, however have a high specificity for the individual organism or the individual species. They can be rapidly identified by the flanking repetitive DNA sequences since they are, to a great extent, statistically distributed over the genome (FIG. 2 (II)), however they aggregate in intergenic segments (FIG. 2 (III)).

### Example 3

### Production of an Organism-Specific Hybridizable Nucleic Acid Molecule

### Exemplified for a PCR Primer

### 1. Biological Sample

As a biological sample the mycelium and sporangia of Peronospora swinglei s.l., the downy mildew of the basil, are provided. “s.l.” means sensu lato, Latin for “in the broader sense”, since the identity of the pathogen is not definitely clear.

### 2. Isolation of the Genomic Information

5 mg of the sporangia are ground in a reaction vial of 2 ml by means of an apparatus adapted therefore, e.g. a shaking mill, by adding milling particles, e.g. small iron beads. 500 μl of a solution of a detergent were added to the ground sporangia to destruct the membranes of the organism and thereby to release the genomic DNA. The solution can e.g. consist of 0.1 M Tris pH 8, 0.05 M EDTA, 2% SDS, proteinase K. The ground sporangia are incubated in the reaction vial with the above-identified solution for several minutes, e.g. 30 minutes, at 65° C. The reaction vial is centrifuged for e.g. 5 minutes at 10 000 s/t²*9.81 to sediment cellular debris.

The liquid phase is removed and transferred into a new reaction vial. A protein-precipitating and preferably phase-separating solution, e.g. chloroform and isoamyl alcohol in a ratio of 24 to 1, for example in the same amount as the liquid already contained in the reaction vial, is added to the liquid phase. The phases are mixed, e.g. by repeated swirling of the reaction vial along its longitudinal axis. The reaction vial is mixed, e.g. for 10 minutes at 10 000 s/t²*9.81 to separate the phases of the protein sedimentation.

The aqueous solution is transferred into a new reaction vial. This is followed by the addition of a DNA precipitating solution, e.g. isopropanol with a volume of 50% of the volume of the transferred aqueous phase. This is followed by an incubation with the DNA precipitating solution, e.g. for 20 minutes at 4° C. The reaction vial is e.g. centrifuged for 20 minutes at 10 000 s/t²*9.81 and 4° C. to precipitate the DNA.

The supernatant is removed. The precipitated DNA is dried, e.g. by blowing with a moderate airflow. The DNA is dissolved in buffer solution or water, e.g. double-distilled water.

### 3. Establishing a Genetic Fingerprint

A PCR is prepared in a reaction vial which is adapted to a PCR, e.g. consisting of 2.5 μl 10×PCR buffer, 2.5 μl of a 2 mM solution contains the four deoxynucleotide triphosphates (dNTPs), 2.5 μl magnesium chloride solution (20 mM), 1 U of a thermostable DNA polymerase, e.g. Taq DNA polymerase, 1 μl of an e.g. 25 mM solution of the PCR primer t3B (5′-AGGTCGCGGTTCGAATCC-3′; SEQ ID no. 36). The primer is designed in such a way that it can bind to the microsatellite t3B. The sample is filled up to 25 μl with water.

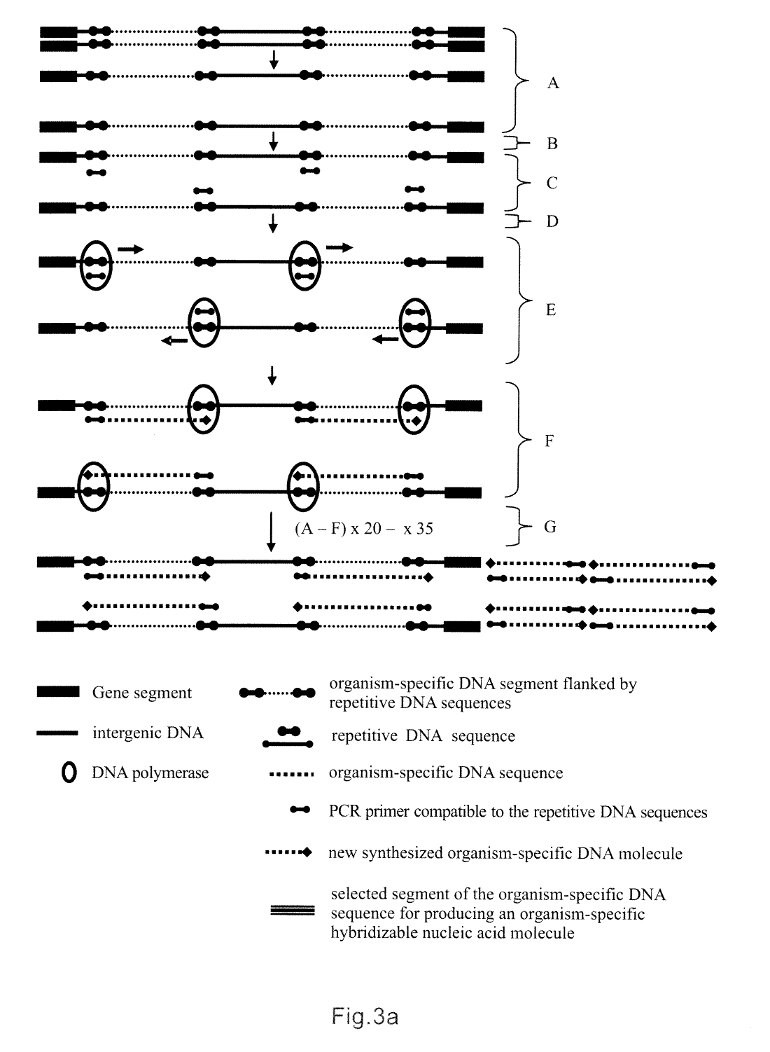

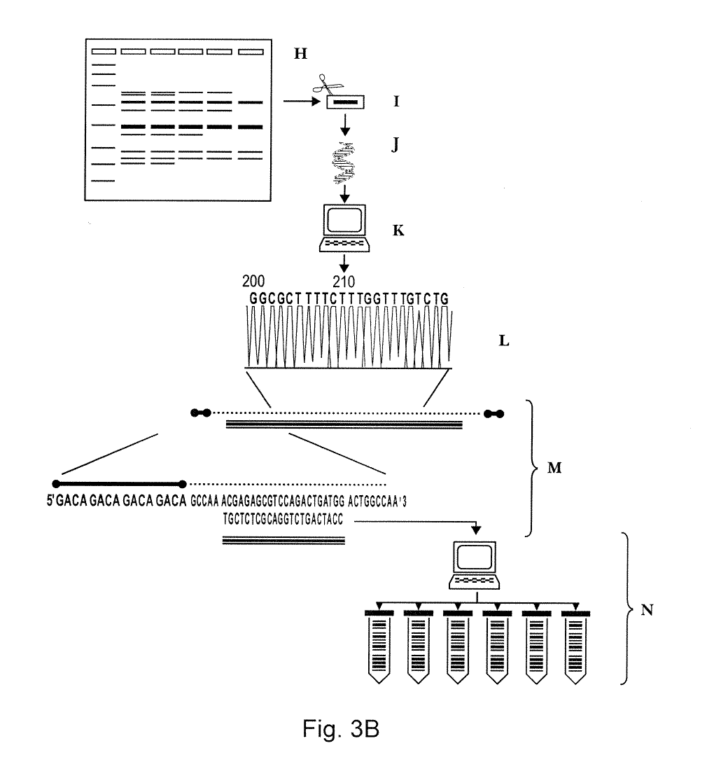

The PCR is performed. The individual steps are schematically shown in FIG. 3.

The incubation in a thermocycler is performed in accordance with the specific modalities of the apparatus. First, e.g. an incubation is performed, e.g. at 94° C. for 2 minutes. In the following an incubation is performed, e.g. at 94° C. for 1 minute. This results in a melting of the DNA double strand; cf. FIG. 3A. In the following the sample is cooled to a temperature, e.g. to 56° C. for 1 minute (FIG. 3B), where the primers can hybridize to the microsatellite t3B; cf. FIG. 3C. In the following the sample is incubated at the working temperature of the polymerase, e.g. at 72° C. for 2 minutes; cf. FIG. 3D. The binding of the polymerase is shown in partial FIG. 3E. New organism-specific fragments which are specific for Peronospora swinglei s.l. are synthesized, which are flanked by repetitive DNA sequences of the t3B microsatellite; cf. FIG. 3F.

The steps A to F of FIG. 3 are subsequently repeated for 20 to 30 times; cf. FIG. 3G.

The amplificates obtained in the PCR which are specific for Peronospora swinglei s.l. are separated, e.g. by means of a gel electrophoresis in an agarose gel; cf. FIG. 3H. This can be e.g. produced by adding 1 g agarose into a receptacle, filling up to 100 μl with an ion-containing liquid, e.g. with Tris-borat-EDTA buffer (TBE). In the following the PCR reaction solution is given into a pre-formed cavity of the agarose gel, e.g. after ⅙ vol. of glycerin and colorants, if applicable, were added, which migrate in the electrophoresis into the same direction as the DNA fragments in the agarose gel and serve as a control of the progress of the electrophoresis. The electrophoresis itself is performed in an adapted electrophoresis chamber which contains an ion-containing solution which is isotonic to the gel, preferably with the same solution which has been used for the preparation of the agarose gel. By applying a voltage, e.g. 140 V, the migration of the DNA fragments is forced through the agarose gel, which are separated by the selective retention which is in particular based on the size of the migrating fragments. In the following a staining with a colorant is performed, which associates or binds to nucleic acids, e.g. ethidiumbromide, e.g. by incubating the agarose gel in a staining bath which contains ethidiumbromide for half an hour.

### 4. Isolation of an Amplificate

A band which was particularly well amplified, in the present case in the region of 400 to 900 bp, is cut out of the agarose gel; cf. FIG. 3I.

The nucleic acid which is specific for Peronospora swinglei s.l., which is contained in this band, is eluted, e.g. by means of a common gel extraction kit, e.g. the QIAquick gel extraction kit, according to the manufacturer QIAGEN; cf. FIG. 3J.

In the following the obtained nucleic acid molecule is inserted into a PCR fragment cloning vector and amplified by means of a commercial PCR fragment cloning kit, e.g. the TOPO TA cloning kit of the company Invitrogen according to the information of the manufacturer.

The vector together with the included nucleic acid fragment which is specific for Peronospora swinglei s.l. are isolated, e.g. by means of a commercial plasmid preparation kit, such as the GeneElute HP Plasmid Midiprep kit of the company Sigma, according to the information of the manufacturer.

### 5. Sequencing of the Isolated Nucleic Acid Molecule

In the following a sequencing of the nucleic acid fragment which is specific for Peronospora swinglei s.l. inserted into the vector, is performed, namely by means of vector specific sequencing primers and common methods, e.g. by means of BigDye v3.1 terminator cycle sequencing kit of Applied Biosystems, according to the information of the manufacturer and subsequent determination of the sequence of the nucleic acid molecule, e.g. by means of a capillary sequencing apparatus, e.g. the apparatus AbiPrism 3730×1 DNA analyzer of Applied Biosystems, according to the information of the manufacturer; cf. FIG. 3K. A part of the chromatogram of the sequencing is shown in partial FIG. 3L.

In the case of this embodiment the nucleotide sequence of SEQ ID no. 2 has been determined as being specific for Peronospora swinglei s.l.

In table 1 several further examples of nucleotide sequences of the genome of different plant pathogens are provided, which are flanked at the 5′ terminus as well as the 3′ terminus by microsatellites such as the t3B, or single sequence repeats (SSRs).

TABLE 1
Examples of nucleic acid molecules or nucleotide sequences,
respectively, which are suitable for the production of an
organism-specific hybridizable nucleic acid molecule.

| | | Highly-variable |
|---|---|---|
| | | fragment |
| Organism | Flanking elements | [SEQ ID no.] |
| Peronospora | Microsatellite t3B; on both sides | 1 |
| arborescens | | |
| Peronospora | Microsatellite t3B; on both sides | 2 |
| swinglei s.l. | | |
| Fusarium | Single sequence repeats (SSRs); on | 3 |
| oxysporum | both sides, sequence: [GACA]₄ | |
| Fusarium | Microsatellite t3B; on both sides | 4 |
| oxysporum | | |
| Plasmopara | Microsatellite t3B; on both sides | 5 |
| petroselini | | |
| Plasmopara | Single sequence repeats (SSRs); on | 6 |
| petroselini | both sides, sequence: [GACA]₄ | |
| Peronospora | Single sequence repeats (SSRs); on | 7 |
| valerianellae | both sides, sequence: [GACA]₄ | |
| Peronospora | Single sequence repeats (SSRs); on | 8 |
| valerianellae | both sides, sequence: [GACA]₄ | |

### 6. Selection of a Segment Suitable for the Production of a Specific Hybridizable Nucleic Acid Molecule

Two segments of the nucleotide sequence of the nucleic acid molecule elucidated in step 5, which is specific for Peronospora swinglei s.l. were selected, which are suitable for the production of PCR primers; cf. FIG. 3M. The nucleotide sequence determined in step 5 provides a matrix for the production of two hybridizable nucleic acid molecules which can be used as PCR primers for the detection of an infection of a plant with Peronospora swinglei s.l.

The nucleotide sequences are selected in such a manner that, according to the state of science, they can specifically hybridize and, according to a data base adjustment (GenBank), are also organism-specific or specific for Peronospora swinglei s.l., respectively, since they can be found as individual parts only in few sequences and in combination in no sequence of a nucleic acid molecule which is catalogized in GenBank.

7. Production of One or Several Nucleic Acid Molecules which Comprise the Segment Selected in Step (6)

The production of the PCR primers which can be used to detect an infection of a plant with Peronospora swinglei s.l. is e.g. realized by means of a 96 column DNA synthesizer workstation of the company PolyGen according to the information of the manufacturer; cf. FIG. 3N. Numerous commercial suppliers, e.g. the company Sigma-Aldrich, offer the preparation of nucleotide polygomers and polymers.

The hybridizable nucleic acid molecule which is designated as S_Ps_F and can be used as forward PCR primer comprises the sequence of 5′-TGCTCTCGCAGGTCTGACTACC-3′ (SEQ ID no. 11). The hybridizable nucleic acid molecule which is designated as S_Ps_R and can be used as backward PCR primer comprises the sequence of 5′-cacgcaaatccctacctctgcc-3′ (SEQ ID no. 12).

Two further hybridizable nucleic acid molecules were designed as primers for an interlaced or nested PCR where a second amplification based on the first amplification is used to further increase the sensitivity. The hybridizable nucleic acid molecule which is designated as S_Ps_Fn and can be used as forward PCR primer comprises the sequence of 5′-GTCTTAACGAGACCGCGTGGTC-3′ (SEQ ID no. 13). The hybridizable nucleic acid molecule which is designated as S_Ps_Rn and which can be used as backward PCR primer, comprises the sequence of 5′-ATGACGGCAGGTGCTGTCGTTC-3′ (SEQ ID no. 14). In the following table 2 several examples of PCR primers are listed, which were generated by means of the method according to the invention.

TABLE 2

Examples of species-specific hybridizable nucleic acid molecules for
the detection of elected plant pathogens.

| Organism specificity | PCR primer | SEQ ID |
|---|---|---|
| [organism] | [sequence] | no. |
| Peronospora arborescens | Forward primer (S_Pa_F): | 9 |
| | 5′-ACGCCCTTTCGGACCAG-3′ | |
| | Backward primer (S_Pa_R): | 10 |
| | 5′-CAGCATGCGCCAGAAGAT-3′ | |
| Peronospora swinglei s.l, | Forward primer (S_Ps_F): | 11 |
| Detection 1 | 5′-TGCTCTCGCAGGTCTGACTACC-3′ | |
| | Backward primer (S_Ps_R): | 12 |
| | 5′-CACGCAAATCCCTACCTCTGCC-3′ | |
| Peronospora swinglei s.l. | Forward primer (S_Ps_Fn): | 13 |
| Detection 2 (nested PCT | 5′-GTCTTAACGAGACCGCGTGGTC-3′ | |
| in connection with | Backward primer (S_Ps_Rn): | 14 |
| detection 1) | 5′-ATGACGGCAGGTGCTGTCGTTC-3′ | |
| Plasmopara halstedii | Forward primer (S_Pl-halst_F): | 15 |
| | 5′-CTC TGT CCA CGC ATT CCG-3′ | |
| | Backward primer (S_Pl-halst_R): | 16 |
| | 5′-GTT TGT ATC TGC TTA AAC TGA C- | |
| | 3′ | |
| Fusarium oxysporum | Forward primer 1 (Detect_Foxy-Psm_F1): | 17 |
| forma specialis strigae | 5′-GGG TTC CCA ATC TCA ATC-3′ | |
| | Forward primer 2: | 18 |
| | 5′-ACT CAC TAC GCC TCG GTC-3′ | |
| | Backward primer (Detect_Foxy-Psm_R): | 19 |
| | 5′-TCG ACA GTC TTT AAG TCT TG-3′ | |
| Plasmopara petroselini | Forward primer (_SPl_petro_F1) | 20 |
| Detection 1 | 5′-ACCAAGAGCGCCTAACTTGACGCT- | |
| | 3′ | |
| | Backward primer (S_Pl_petro_R1) | 21 |
| | 5′-GGGTTCGAATCCGGGGCATCTCGT- | |
| | 3′ | |
| Plasmopara petroselini | Forward primer (S_Pl_petro_F1n) | 22 |
| Detection 2 (nested PCT | 5′-CCGTCAACTCATAGGCGAATGCA-3′ | |
| in connection with | Backward primer (S_Pl_petro_R1n) | 23 |
| detection 1) | 5′-CACTAAACCAACGGCGCTTGCACG- | |
| | 3′ | |
| Plasmopara petroselini | Forward primer (S_Pl_petro_F2) | 24 |
| Detection 3 | 5′-CGCATTATCGCTCTAGGAAGCGAC- | |
| | 3′ | |
| | Backward primer (S_Pl_petro_R2) | 25 |
| | 5′-GGCCCGAATGTGATGTGCCGCGTT- | |
| | 3′ | |
| Plasmopara petroselini | Forward primer (S_Pl_petro_F2n) | 26 |
| Detection 4 (nested PCT | 5′-CCAGCTACGCACATGGAACGACCT- | |
| in connection with | 3′ | |
| detection 3) | Backward primer (S_Pl_petro_R2n) | 27 |
| | 5′-ACGCAAGAATCATACCTCGCTGC-3′ | |
| Peronospora valerianel- | Forward primer (S_Per_val_F1) | 28 |
| lae | 5′-GACAGATTGCGTTACCCACTGGCT- | |
| Detection 1 | 3′ | |
| | Backward primer (S_Per_val_R1) | 29 |
| | 5′-CCGACAAAACGTGTTGCTGCTGCA- | |
| | 3′ | |
| Peronospora valerianel- | Forward primer (S_Per_va_F1n) | 30 |
| lae | 5′-TTGCGTCGACGTTGGCGATGCACG- | |
| Detection 2 (nested PCT | 3′ | |
| in connection with | Backward primer (S_Per_val_R1n) | 31 |
| detection 1) | 5′-GAGCGCTCTAAGTGAGTGTCTCGT- | |
| | 3′ | |
| Peronospora valerianel- | Forward primer (S_Per_val_F2) | 32 |
| lae | 5′-GACGTACAAATCACCCCGATGTCG- | |
| Detection 3 | 3′ | |
| | Backward primer (S_Per_val_R2) | 33 |
| | 5′-TGCTGACGTTCACAAGCGCTA-3′ | |
| Peronospora valerianel- | Forward primer (S_Per_val_F2n) | 34 |
| lae | 5′-GATACTCCGGCATGACAGTGTCGT- | |
| Detection 4 (nested PCT | 3′ | |
| in connection with | Backward primer (S_Per_val_R2n) | 35 |
| detection 3) | 5′-GGGATTTGCGGTAACCCTGTCAGC- | |
| | 3′ | |

### Example 4

### Use of the Nucleic Acid Molecule According to the Invention (PCR Primer) for the Specific Detection of Peronospora swinglei s.l. in Seeds of Basil

The detection method essentially comprises the following 5 working steps: (a) disintegration of the seeds; (b) DNA extraction; (c) quality testing of the obtained DNA solution; (d) application of the PCR-based detection and (e) electrophoretic separation of the PCR reaction solutions.

### (a) Disintegration of the Seeds

To detect an internal and external contamination with the pathogen basil seeds of seven different parts of the seeds were mechanically disintegrated in liquid nitrogen for 5 min before the extraction. Alternatively also other disintegration technologies can be used, such as grinding in mills, mixers and enzymatic degradation of structure-giving components of the cell.

In the following 5 g (Ø 1000 grain weight of basil seeds) of the ground seeds were transferred into a sterile 50 ml centrifugation tube.

(b) DNA Extraction (According to Möller et al. 1992, Modified)

After thoroughly suspending in 20 ml SSB buffer (Tris: 200 mM, NaCl: 300 mM, EDTA: 25 mM, SDS: 1% (v/w)) the mixture was incubated for 30 min at room temperature with gentle shaking. After the sedimentation of the insoluble components by a centrifugation for five minutes at minimum 9.000 s/t2*9.81, 5 ml of the supernatant were removed and transferred into a new centrifugation tube of 50 ml. The remaining 15 ml of the SSB buffer were almost completely absorbed by the strongly swelling ingredients of the basil seeds. After the addition of 625 μl of a 4 M potassium acetate solution the extraction solution was incubated for 30 min on ice with gentle shaking. After the sedimentation of the insoluble components by a centrifugation at minimum 9.000 s/t2*9.81, the liquid supernatant was completely transferred into a new centrifugation tube. After 0.6 volume portions of isopropanol (100%) the extraction solution was incubated for 10 minutes at room temperature with gentle shaking. The insoluble DNA was sedimented by a centrifugation at minimum 9.000 s/t2*9.81. After a complete removal of the supernatant the sediment was washed two times with ethanol (70%) and was subsequently dried.

The dried sediment was resuspended in 5 ml TE buffer and subsequently 500 μl of a 1% spermidine were added. After an incubation for 15 minutes on ice the insoluble components were sedimented by a centrifugation for 10 minutes, the supernatant was discarded. After the resuspension of the sediment by adding 1 ml spermidine removal buffer (sodium acetate: 300 mM; magnesium acetate: 10 mM) and 3 ml cold ethanol (100%) an incubation phase of 1 hour on ice takes place with simultaneous gentle shaking. The insoluble DNA is sedimented by a centrifugation for 10 minutes at minimum 9.000 s/t2*9.81. After a complete removal of the supernatant the sediment was washed two times with ethanol (70%) and was subsequently dried.

At the end 500 μl H₂O were added to the isolated and purified DNA, transferred into a 1.5 ml reaction vial and stored at −18° C. until the further use.

### (c) Quality Testing of the Obtained DNA Solution

### Measurements:

The concentration and the purity of the isolated DNA molecules were determined by means of the absorption measurement. The determination of the concentration results from the measured value of the optical absorption in the UV region at 260 nm. The purity of the isolated DNA was determined from the ratio of the absorption measurement at 260 and 280 nm (BioPhotometer, Eppendorf, Germany). In parallel the quality of the isolated DNA was evaluated by electrophoretic separation in an agarose gel (1%).

In the case of significant impurities the DNA was post-purified by means of a commercial DNA purification kit, as e.g. provided by GE Healthcare in the form of the “illustra GFX™ PCR DNA and gel band purification kit” and again tested.

### Control PCR:

To test the capability of the obtained DNA for an amplification a PCR with primers for a portion of the chloroplast DNA of basil was performed. Only DNA solutions which resulted in a definite amplificate in the agarose gel of the control PCR with the size of 520 nt are allowed for the PCR-based detection of the pathogen. By this procedure wrong negative results can be excluded.

### (d) Application of the PCR-Based Detection

### Detection Reaction 1

A PCR reaction was prepared in a reaction vial which is adapted for a PCR, e.g. consisting of 2.5 μl 10×PCR buffer, 2.5 μl of a 2 mM solution of the four deoxynucleotide triphostates (dNTPs), 2 μl magnesium chloride solution (20 mM), 1 U of a thermostable DNA polymerase, e.g. Taq DNA polymerase, 1 μl of a e.g. 25 mM solution of the PCR primers S_Ps_F (5′-TGCTCTCGCAGGTCTGACTACC-3′; SEQ ID no. 11) and S Ps R (5′-CACGCAAATCCCTACCTCTGCC-3′; SEQ ID no. 12). The primers are designed in such a way that they can bind to the identified intergenic DNA section (SEQ ID no. 2) which is specific for Peronospora swinglei s.l. The sample is filled up with water to 25 μl.

In the following a PCR is performed, e.g. under the following conditions: The incubation in a thermocycler is performed according to the modalities of the apparatus. First, e.g. an incubation is performed at 94° C. for 5 min. In the following an incubation at e.g. 94° C. for 30 sec is performed. Then an incubation at 65° C. for 30 sec. In the following an incubation at 72° C. for 40 sec. The last three steps are subsequently repeated e.g. for 45 times. Then a final incubation at 72° C. for 4 min can be performed.

### Detection Reaction 2

A second detection reaction (nested PCR) follows. For this 1 μl of the first detection reaction and 1 μl of an e.g. 25 mM solution of the nested primers S_Ps_Fn (5′-GTCTTAACGAGACCGCGTGGTC-3′ SEQ ID no. 13) and S_Ps_Rn (5′-ATGACGGCAGGTGCTGTCGTTC-3′ SEQ ID no. 14) were added to the remaining solutions and reagents (10×PCR buffer, dNTPs, magnesium chloride solution and Taq DNA polymerase) into a new reaction vial which is adapted to a PCR. The incubation in a thermocycler is performed in accordance with the modalities which are specific for the apparatus. First, e.g. an incubation at 94° C. for 5 minutes is taking place. In the following an incubation at e.g. 94° C. for 30 sec. Subsequently an incubation at 65° C. for 30 sec. In the following an incubation at 72° C. for 30 sec is taking place. In the following the last three steps are repeated for e.g. 35 times. Then a final incubation at 72° C. for 4 minutes can be performed.

### (e) Electrophoretic Separation of the PCR Reaction Solutions

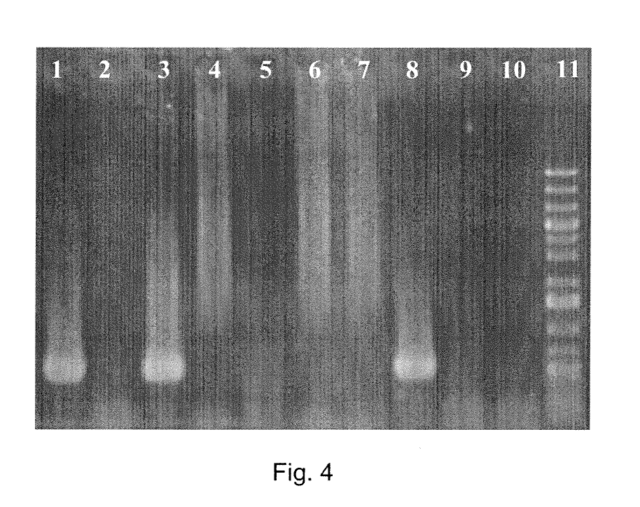

The amplificates obtained in the detection reaction 2 were separated, e.g. by means of a gel electrophoresis, e.g. in an agarose gel. This can e.g. be prepared by adding 1 g agarose into a receptacle, filling up to 100 μl with an ion-containing liquid, e.g. with Tris-borat-EDTA buffer (TBE). In the following the PCR reaction solution is deposited into a preformed cavity of the agarose gel, e.g. after the addition of ⅙ vol. glycerin and colorants, if applicable, which migrate in the electrophoresis in the same direction as the DNA fragments in the agarose gel and serve to control the progress of the electrophoresis. The electrophoresis itself is performed in an adapted electrophoresis chamber which is filled with an ion-containing solution which is isotonic to the gel, preferably with the same solution which was used for the preparation of the agarose gel. By applying a voltage, e.g. 140 V, the migration of the DNA fragments through the agarose gel is forced, which are separated by the selective retention which is in particular based on the size of the migrating fragments. In the following a staining with a colorant which associates or binds to nucleic acids, e.g. ethidiumbromide, is performed, e.g. by incubating the agarose gel for half an hour in an ethidiumbromide-containing staining bath. The result is shown in FIG. 4.

The lanes of the 1.5% agarose gel were as follows: lane 1: seed sample 1 (contaminated); lane 2: seed sample 2; lane 3: seed sample 3 (contaminated); lane 4: seed sample 4; lane 5: seed sample 5; lane 6: seed sample 6; lane 7: seed sample 7; lane 8: reference DNA of Peronospora swinglei s.l (positive control); lane 9: reference DNA of Ocimum basilicum (basil) (negative control 1); lane 10: PCR preparation without DNA (negative control 2); lane 11: DNA size marker (standard) 1.

The reference sample of Peronospora swinglei s.l. (lane 8) shows a clear visible amplificate with a size of 500 nt (A). The white band is the organism-specific hybridizable nucleic acid molecule of Peronospora swinglei s.l. The same amplificate can be found in the PCR preparations of the seed samples 1 (lane 1) and 3 (lane 3), no amplificate can be found in the seed samples 2, 4-7 (lanes 2, 4-7) and in the negative controls 1 and 2 (lanes 9 and 10).

Therefore, by the method according to the invention the pathogen Peronospora swinglei s.l. could be clearly identified in the seed samples 1 and 3. The appearing smear which can be found in lanes 4, 6 and 7 is an unspecific side reaction which is typical for nested PCR preparations, and is not to be confused with the specific amplificate in the lanes 1, 3 and 8. Wrong negative reactions can be excluded by the foregoing quality test. Wrong positive reactions are excluded by the co-processed negative controls 1 and 2.

As shown in the practical example, the application of the method according to the invention in form of a PCR-based detection leads to a definite interpretable result. The seeds which were contaminated by the pathogen Peronospora swinglei s.l. could be identified in a reliable manner and removed from the market. As a result, the distribution of the target organism, in this case of a dangerous pathogen, is hereby prevented.

TABLE 3
Comparison of a classical DNA fingerprinting with
the detection method according to the invention

| | | | Method according to the |
|---|---|---|---|
| | | | invention |
| | | | Organism-specific |
| | | Pattern analysis | hybridizable nucleic acid |
| | | Classical DNA finger- | molecules (e.g. PCR |
| | Principle of detection | printing | primers) |
| 1 | Organism must be present in | Yes | No |
| | a pure culture | | |
| 2 | Processing time | 3 days to several weeks | 2 days |
| 3 | Interpretation of the rest | Difficult and time- | Simple (yes/no answer) |
| | results | consuming | |
| 4 | Comparability of the test | Only to a limited extent | Yes |
| | results | | |
| 5 | Handling | Complex | Simple |
| 6 | Training effort | Much | Few |
| 7 | Realizable as a kit | Only to a limited extent | Yes |
| 8 | Developmental period | Very long | short |
| 9 | Genome-wide screening | Yes | Yes |
| 10 | Differentiation between | Possible | Possible |
| | closely-related species | | |
| 11 | Usable as probes | No | Yes |
| 12 | Identification of new differentiating | Extremely complex | Easy and rapidly possibly |
| | DNA segments | | |

As demonstrated in table 3 the detection method according to the invention has numerous advantages over the pattern analysis. In particular it is characterized by its flexible use (lane 11) and its high adaptability to new organisms (lane 12). With the production method according to the invention the user is in the position to develop new organism-specific detection methods within a short time and to use them in practice for a reliable identification of organisms.

1. Method for producing an organism-specific hybridizable nucleic acid molecule, comprising the following steps:
1. Providing a genomic information originating from an organism;
2. establishing a genetic fingerprint from the genomic information to obtain a pattern of amplificates of genome segments by the following steps:
2.1 amplifying segments of the genomic information of the organism to obtain a mixture of amplificates, and
2.2 separating the amplificates contained in the mixture to obtain a pattern of amplificates of genome segments;
3. isolating at least one amplificate from the pattern;
4. sequencing the isolated amplificate to obtain a nucleotide sequence;
5. selecting a segment of the nucleotide sequence of 3 to 1000 nucleotides suitable for producing an organism-specific hybridizable nucleic acid molecule, and
6. producing a nucleic acid molecule comprising the segment selected in step (5).

2. Method according to claim 1, wherein the genomic information of the organism is double-stranded DNA.

3. Method according to claim 2, wherein the amplification in step 2.1 is performed by a polymerase chain reaction (PCR).

4. Method according to claim 3, wherein in the context of the PCR such a forward primer is selected, which hybridizes to a nucleotide sequence of a satellite DNA on the first DNA strand of the organism, and such a backward primer is selected, which hybridizes to a nucleotide sequence of a satellite DNA on the DNA strand of the organism complementary to the first DNA strand.

5. Method according to claim 4, wherein the satellite DNA is selected from the group consisting of: microsatellite DNA, simple sequence repeats (SSRs) and intersimple sequence repeats (ISSRs).

6. Method according to claim 1, wherein the separating of the amplificates contained in the mixture is performed by means of a chromatographic and/or electrophoretic method.

7. Method according to claim 1, wherein from the pattern of amplificates of fragments of the genomic information such an amplificate is isolated which is, in comparison to the remaining amplificates, amplified in an amount beyond average.

8. Method according to claim 1, whereinafter step (3) and before step (4) the following further steps are performed:
a) inserting the isolated amplificate into a vector,
b) introducing the vector into an appropriate organism, and
c) isolating the vector from the organism to obtain at least one isolated amplificate.

9. Method according to claim 1, wherein the selected segment suitable for producing an organism-specific hybridizable nucleic acid molecule comprises a length of 10 to 64 nucleotides.

10. Method according to claim 1, characterized in that the produced nucleic acid molecule is designed as a PCR primer.

11. Nucleic acid molecule comprising any of the nucleotide sequences of SEQ ID No. 1 to 35 or a nucleotide sequence complementary thereto.

12. Nucleic acid molecule which hybridizes under stringent conditions to the nucleic acid molecule according to claim 11.

13. Method for detecting an organism in a biological sample, comprising the following steps:
1. providing a biological sample containing nucleic acid,
2. isolating the nucleic acid from the sample,
3. subjecting the isolated nucleic acid to a polymerase chain reaction (PCR),
4. determining whether in step 3 an amplification of the isolated nucleic acid has taken place, and
5. correlating a positive determination in step 4 with the presence of the organism in the sample, and correlating a negative determination in step 4 with the absence of the organism in the sample,
wherein in step 3 as a PCR primer at least one nucleic acid molecule is used which was obtained by the method according to claim 1.

14. Method according to claim 13, wherein as the PCR primer at least one nucleic acid molecule comprising any of the nucleotide sequences of SEQ ID No. 9 to 35 or a nucleotide sequence complementary thereto is used.

15. Method according to claim 13, wherein the PCR reaction in step 3 is designed as a nested PCR reaction.


SEQUENCE LISTING (39 sequences)

SEQ ID NO: 1 - DNA, 812 bp, Peronospora arborescens
cggccgccagtgtgctggaattcgcccttaggtcgcgggttcgaatccacgccctttcggaccagtgccttaaactggcgccttagaccgctcggccaattgaccgtcaaccacaaaccacaaccctcaaccgtacacaaaacatacacaaaccacaacaactcattacaactcaacgcacacctcgctcgccaaatacacagtcgacacgtgcaagaactggccaaacatgccgccacccttctccacaactcgctccaccatcgtttctgtctgcttacgatgccgtcggcaagaactgggtgactgcctgtcgacagcatagcgggcctttatgctgctgtagtagaagtgatgcacgattgtactgaaggcaccacgcactaagctatgaccaccacaccaaatcgatcgatcttcgtggcgagccagagtgacctacatctgtcagtccctgcatcagtccgggatgagtccagcctcgcatcttctggcgcatgctgctcacgataacaagtaaaccaactattacctctactgacagcagcggatatgcagcttatagtgtagcacagtcgaggagcagcgtccgaagtgcagcaacgtgaatttgacaaaggtttgatatcaaaagcaggtgtagtcgccggacttgagcccacgcgaaagcaagtagtcagttagctggtccctccggtgtgacatagctcaatcaacagcatcaaagacgcgtgttcgcgagatataggggaatggctggagcataatagaaaagaatgagttgcccaacctccttgggattcgatacccgc

SEQ ID NO: 2 - DNA, 784 bp, Peronospora swinglei s.l.
gtcgtcgtcgtcgtccatctcaatgctggacatggctgctctcgcaggtctgactaccatgaacgatgcagtatctccaactggatcaacctactcgacttcctcttccatgtcgtactcgtcactggcttcacgtgatgtcatgtacacacccgactcggtcgactgcagtcttaacgagaccgcgtggtctggtctacccgttggcggtgcttcgtacttgatcgagtttgacgacatgcccctatctatgagcagccaccagagcttcttatcttcagacgagatcgagtttctgtgctctgccatccttgagtaattcgcaagcacaagaagcaaaacgcctccctcagcattctaagaggtcatgcaaacaagcttttgttccgtcgaacccggttggcaagtgctgctgtcggcttcaatctctcgagcgtatagtacgtagcacattaagtttagagcattataattcttcattgtagacttcgttgtataaagtcataatggaagggatatctatctatcaacaaaaagcttgcaacctttcgtattgaacgctatttgatttcattggcgactaatgctagcgaggtatcattgctaccagtagagccactggtttgtctaaccgatagcagccgtcaatgaacgacagcacctgccgtcatgaggacccactcgttgaagtctccatggcatataaaaaacgctaccatgaccaatccaacaggaccagcctcatcttgaaattggcagaggtagggatttgcgtgcaccggac

SEQ ID NO: 3 - DNA, 619 bp, Fusarium oxysporum
aagggaacaaaagctgggtaccgggccccccctcgaggtgacggtatcgataagcttgatatccactgtggaattcgcccttgacagacagacagacagctcacggccaacagcatacaggaaatttgtttcttgcagcgttgccatggcctggtatcccccccacacaatccttctcaataatagctgtttaggtacaccctcctccttcacgggactttcaaacgggttccccaatctcaatcccgagaccttctcgactcagtacgcctcggtctctttctagatgttatctgaacaagcttyaattgctctttgagctcctatgcatagttaggatcattgcctttgataaatctatctctggggcaccgacctcgatttcattgagccgctgggtggccatgtccgaactctttgacaaatctttataaatatcacctatgttaacagtaccaagcgcaagacttgaaagactgtcgagggttgtgctgctgttttcattcacatctttccggtccttgtctgtctgtctgtcaagggcgaattccacattgggctgcagcccgggggatccactagttctagagcggccgcaccgcgggagctccaattcgcc

SEQ ID NO: 4 - DNA, 782 bp, Fusarium oxysporum
  misc_feature (115)..(115): n is a, c, g, or t
aagctgggtaccgggccccccctcgaggtcgacggtatcgataagcttgatatccactgtggaattcgcccttaggtcgcgggttcgaatccccataggtatgtgttatctgacnagggggcctattctcttatccctcttactgattttgatggtgaagcttgcgatgcatcgcgtggaatccgctcgggactctagtagcaacggggtcgtcagagaaaaccctcagagtgtgtaagttgatcaagtctgaaggcctgtccggccactagagcgaagctgaatgagatagggaaccctgaaaagcctcatgtgaagttttcaacggagctcaagggccactcggcatctgttgagaaggtcgcattcaacccaacaaaagatgcagagctttgcagtgtgagcagtgatggcgtggtcaagttctgggacgtgaggacaaaagcatgcttcaatgaggtcaaaggccttggggacgcttttactcttgtctgggcaccagatggaaacacgcttctcgttgggaacaaggcagataccatcttcgttctgtngccatctcagtcgacgcctctcagcacgcatcagcagtccgtgcagaccaaccagatcgcattctgntggagcggtgagaagatcttcctcacgacagctgaaggaagtattcgagtcttgtcgtaccctggattagaacccgcgacctaagggcgaattccacattgggctgcagcccgggggatccactagttctagagcggccgcaccgcgggagctccaattcg

SEQ ID NO: 5 - DNA, 231 bp, Plasmopara petroselini
accaagagcgcctaacttgacgcttgtctgaaatccgaccgtcaactcataggcgaatgcaaatttagtttaccagtatttgatcatcatacatggcgtgtaagacgtctaataatgaagacgcaagattgtggagcgtaaggagtccttttctcgtccgtgcaagcgccgttggtttagtggtaaaatatctcgttgccaacgagatgccccggattcgaacccgcgacc

SEQ ID NO: 6 - DNA, 691 bp, Plasmopara petroselini
atgtagaagaagacaggcaggcagactcataagtaaatgcgcattatcgctctaggaagcgacactactgtacattttcccgsacctatatacatacatgcacatactacacagtgccagctacgcacatggaacgacctagattaccttacccttcttgccaatctcgcctccctagtcattggccagtgacaggtgaccagtggctcggatctgcatcgccacgatgcttggtatgccacatgcataatctgcgacacatggccgccccatggcttggtgtatggcttggtgttcccccatgcaagccatagccgtcacgttgctgtgctctggtcacaatcgagatgttccaggggcaagtaaacacgcggctatgcggctcgaaaagctcgcctaagcaaagcgccacgcaaagtctggaggagcgctgaacggaagaggcggcgacggcagtccactacacttcgagaaaatgaattttagctttggtgcccggcagatgaggcgcttccacactttcgagaggctccgcggctacgtctagttcgttagggcccgtatacgagtcggaagggcagcgaggtatgattcttgcgtgcaccggtgcagtgctaaacgcggcacatcacattcgggcctagcgacgaaggcaatagtgcgaaaaaggttgccgctcccgtctcgcccg

SEQ ID NO: 7 - DNA, 317 bp, Peronospora valerianellae
gacagattgcgttacccactggctaaaaagaatacaagacatacgaaagacatgacagttgactcactctctctatgtgtgtgtgtgatttcgttggtgtagatttctcgtgtgccattgcgtcgacgttggcgatgcacggacgcatgtatccaacgagttcccacgtgtgtttctactcgaatgtgtttggacgagagagaaaggtcaggaaggtagcgaggttgaaccttgacgagacactcacttagagcgctctcttgggatttgcagcagcaacacgttttgtcggcttttggtgtaatgttgatctgttg

SEQ ID NO: 8 - DNA, 463 bp, Peronospora valerianellae
gaatccgacgttttgggaggtctagaggcagcgacgtacaaatcaccccgatgtcgcgtgaatgctgtccatatatgctggacactggcttgggctagtgttgtacaaagatactccggcatgacagtgtcgtgagacgagttcggcaatgctcaccagattgttcgacggaaaatacgacggagccgtgtgcttacgatctcagaccttgatacccgtactactactgtctcgagactgtaccatcgatctaggtcgaccagaccaagcgaagctcatgtctgtgtaacttgggcgagagcattcccgctggagcagcattgttgaagctcctcgccaggtagtagcactcgctgttcatttcacgctgacagggttaccgcaaatccctcagacggcacacaaaagcgcgcaacgaatgatagcgcttgtgaacgtcagcagaagcacggcgtcgcaacca

SEQ ID NO: 9 - DNA, 17 bp, Artificial sequence
  Synthetic Oligonucleotide
acgccctttcggaccag

SEQ ID NO: 10 - DNA, 18 bp, Artificial sequence
  Synthetic Oligonucleotide
cagcatgcgccagaagat

SEQ ID NO: 11 - DNA, 22 bp, Artificial sequence
  Synthetic Oligonucleotide
tgctctcgcaggtctgactacc

SEQ ID NO: 12 - DNA, 22 bp, Artificial sequence
  Synthetic Oligonucleotide
cacgcaaatccctacctctgcc

SEQ ID NO: 13 - DNA, 22 bp, Artificial sequence
  Synthetic Oligonucleotide
gtcttaacgagaccgcgtggtc

SEQ ID NO: 14 - DNA, 22 bp, Artificial sequence
  Synthetic Oligonucleotide
atgacggcaggtgctgtcgttc

SEQ ID NO: 15 - DNA, 18 bp, Artificial sequence
  Synthetic Oligonucleotide
ctctgtccacgcattccg

SEQ ID NO: 16 - DNA, 22 bp, Artificial sequence
  Synthetic Oligonucleotide
gtttgtatctgcttaaactgac

SEQ ID NO: 17 - DNA, 18 bp, Artificial sequence
  Synthetic Oligonucleotide
gggttcccaatctcaatc

SEQ ID NO: 18 - DNA, 18 bp, Artificial sequence
  Synthetic Oligonucleotide
actcactacgcctcggtc

SEQ ID NO: 19 - DNA, 20 bp, Artificial sequence
  Synthetic Oligonucleotide
tcgacagtctttaagtcttg

SEQ ID NO: 20 - DNA, 24 bp, Artificial sequence
  Synthetic Oligonucleotide
accaagagcgcctaacttgacgct

SEQ ID NO: 21 - DNA, 24 bp, Artificial sequence
  Synthetic Oligonucleotide
gggttcgaatccggggcatctcgt

SEQ ID NO: 22 - DNA, 23 bp, Artificial sequence
  Synthetic Oligonucleotide
ccgtcaactcataggcgaatgca

SEQ ID NO: 23 - DNA, 24 bp, Artificial sequence
  Synthetic Oligonucleotide
cactaaaccaacggcgcttgcacg

SEQ ID NO: 24 - DNA, 24 bp, Artificial sequence
  Synthetic Oligonucleotide
cgcattatcgctctaggaagcgac

SEQ ID NO: 25 - DNA, 24 bp, Artificial sequence
  Synthetic Oligonucleotide
ggcccgaatgtgatgtgccgcgtt

SEQ ID NO: 26 - DNA, 24 bp, Artificial sequence
  Synthetic Oligonucleotide
ccagctacgcacatggaacgacct

SEQ ID NO: 27 - DNA, 23 bp, Artificial sequence
  Synthetic Oligonucleotide
acgcaagaatcatacctcgctgc

SEQ ID NO: 28 - DNA, 24 bp, Artificial sequence
  Synthetic Oligonucleotide
gacagattgcgttacccactggct

SEQ ID NO: 29 - DNA, 24 bp, Artificial sequence
  Synthetic Oligonucleotide
ccgacaaaacgtgttgctgctgca

SEQ ID NO: 30 - DNA, 24 bp, Artificial sequence
  Synthetic Oligonucleotide
ttgcgtcgacgttggcgatgcacg

SEQ ID NO: 31 - DNA, 24 bp, Artificial sequence
  Synthetic Oligonucleotide
gagcgctctaagtgagtgtctcgt

SEQ ID NO: 32 - DNA, 24 bp, Artificial sequence
  Synthetic Oligonucleotide
gacgtacaaatcaccccgatgtcg

SEQ ID NO: 33 - DNA, 21 bp, Artificial sequence
  Synthetic Oligonucleotide
tgctgacgttcacaagcgcta

SEQ ID NO: 34 - DNA, 24 bp, Artificial sequence
  Synthetic Oligonucleotide
gatactccggcatgacagtgtcgt

SEQ ID NO: 35 - DNA, 24 bp, Artificial sequence
  Synthetic Oligonucleotide
gggatttgcggtaaccctgtcagc

SEQ ID NO: 36 - DNA, 18 bp, Artificial sequence
  Synthetic Oligonucleotide
aggtcgcggttcgaatcc

SEQ ID NO: 37 - DNA, 749 bp, Artificial sequence
  Viral genome sequence
aattccaatatctttgtgaaccgtttacttagtttttcgtttgtttttttaataatataatatcgttcgtttccaaacaatttttcaatattttttggattaatatattatataattcgaaatatttttggttccaataaacgaaattcttttggtatttataaaaaaagaatttcccgttttctttatattggttaataattttaaatatttatatttttcgttaaattattattattatttccggatttttaaaagacccgtaaaaatgtttcgatatttttttaaaaaacgccggtaattttaatattcgtcgtcgttttggaaatccaatttctgtcacagacctgaaggacagccactgggctgtcgcttagtcatgacccctgtcacgtgaaggcgcgagggccgagcgcctcgcgcgcgtatatctacgcgaaatctctgcagtctccgatatgtagctcctcgagctacgtcttcgtcgacaaccacctgctacactgtacctggaagactagatagccaatatactctgtcctgttacctgatcgcccgcacctacctgtccgccctgcctgcccgtgacattacgtagctccttcattaggtgcccgcgatgcctgcgtcactgcaacccccgatcttaaccgattcgaccgaggaactgatcgaacacctacaaggacaccctgaacaatgggtgtcttacatctccgattcctaccaaaagctgcaactattgcacg

SEQ ID NO: 38 - DNA, 751 bp, Artificial sequence
  Repetitive chromosomal sequence
ggatccactctgtccacgcattccggcagctttgagttttggttagtggtttccagcatcgatgggtaccaccatcgcatttgaatcgatattgctgtcatttgagcctatgcgagcacttgtatcgcaatgtcagacaccaatgtccatacagcaacgtcgcttcaaatctatgcgaccacttgtttgcgtttcattgtgaagcatcgttagacaaatcaactttgtccatgcattcttgcagcttgggaaatgcttagttgttatcaatctccatgtttagcatctccaggacaaacacaacgtgtagcattaccaggccaaagtaaatgttcataagcaatagccgttgctaacagcttaaataaatgcaaacacttgttggcagttcactgtttagcgtctttaagcaaaaccactttgttcatgcatcctcaaagccgtgaacatgctatgtggttggcagtgtccaacttttgcatctccaagccaatcacaataatcattagcaatcgccattgctatcagcccgattagatgcaaacacttcgcagtcactgtaaagcgtctttgagcgaatttacattgtccatgcatcctcacagtcatgaacagtttaagtggttgccagcgttcatgtgtagaagcactagaccaaatataatgttcatcagcaatcgccaatgctgtcagtttaagcagatacaaacacttgtttggtgtcgactgttaagcgactttgaacggatcc

SEQ ID NO: 39 - DNA, 15 bp, Artificial sequence
  Synthetic Oligonucleotide
gtcgtcgtcgtcgtc